SEQUENCE LISTING (12 sequences)

SEQ ID NO: 1 - DNA, 378 bp, Homo sapiens
atgcacaaggaggaacatgaggtggctgtgctggggccaccccccagcaccatccttccaaggtccaccgtgatcaacatccacagcgagacctccgtgcccgaccatgtcgtctggtccctgttcaacaccctcttcttgaactggtgctgtctgggcttcatagcattcgcctactccgtgaagtctagggacaggaagatggttggcgacgtgaccggggcccaggcctatgcctccaccgccaagtgcctgaacatctgggccctgattctgggcatcctcatgaccattggattcatcctgttactggtattcggctctgtgacagtctaccatattatgttacagataatacaggaaaaacggggttactag

SEQ ID NO: 2 - DNA, 378 bp, Homo sapiens
atgcataaggaagaacacgaggtggccgtgctgggacctccacctagcacaattctgcctagaagcaccgtgatcaacatccacagcgagacaagcgtccccgaccacgtggtgtggtccctgttcaacaccctgttcctgaactggtgctgtctgggatttatcgccttcgcctacagcgtgaaaagcagagatagaaagatggtgggcgacgtgaccggcgcccaggcttatgcttctaccgccaagtgcctgaatatctgggccctgatcctgggcatcctgatgaccatcggcttcatcctcctgctggtgttcggctctgttacagtgtaccacatcatgctgcaaatcatccaggagaagcggggctactga

SEQ ID NO: 3 - protein, 125 aa, Homo sapiens
MHKEEHEVAVLGPPPSTILPRSTVINIHSETSVPDHVVWSLFNTLFLNWCCLGFIAFAYSVKSRDRKMVGDVTGAQAYASTAKCLNIWALILGILMTIGFILLLVFGSVTVYHIMLQIIQEKRGY

SEQ ID NO: 4 - DNA, 402 bp, Homo sapiens
atgaatcacactgtccaaaccttcttctctcctgtcaacagtggccagccccccaactatgagatgctcaaggaggagcacgaggtggctgtgctgggggcgccccacaaccctgctcccccgacgtccaccgtgatccacatccgcagcgagacctccgtgcccgaccatgtcgtctggtccctgttcaacaccctcttcatgaacccctgctgcctgggcttcatagcattcgcctactccgtgaagtctagggacaggaagatggttggcgacgtgaccggggcccaggcctatgcctccaccgccaagtgcctgaacatctgggccctgattctgggcatcctcatgaccattctgctcatcgtcatcccagtgctgatcttccaggcctatggatag

SEQ ID NO: 5 - DNA, 402 bp, Homo sapiens
atgaatcacaccgtgcagacctttttcagccctgtgaacagcggccagcctccaaactacgagatgctgaaggaagaacacgaggtggccgtgctgggagcccctcataatcctgctcctcctaccagcaccgtgatccacatccggagcgagacatctgtccccgaccacgtggtgtggtccctgttcaacaccctgtttatgaacccctgctgtctgggcttcatcgccttcgcctacagcgtgaaaagcagagatagaaagatggtgggcgacgtgaccggcgcccaggcctacgcctctacagccaagtgcctgaacatctgggccctgatcctgggcatcctcatgacaatcctgctgatcgtgatccccgtcctgattttccaagcttatggctga

SEQ ID NO: 6 - protein, 133 aa, Homo sapiens
MNHTVQTFFSPVNSGQPPNYEMLKEEHEVAVLGAPHNPAPPTSTVIHIRSETSVPDHVVWSLFNTLFMNPCCLGFIAFAYSVKSRDRKMVGDVTGAQAYASTAKCLNIWALILGILMTILLIVIPVLIFQAYG

SEQ ID NO: 7 - DNA, 1497 bp, Homo sapiens
atgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttga

SEQ ID NO: 8 - DNA, 1497 bp, Homo sapiens
atgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttag

SEQ ID NO: 9 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 10 - DNA, 204 bp, Cytomegalovirus
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 11 - DNA, 304 bp, Cytomegalovirus
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 12 - DNA, 508 bp, Cytomegalovirus
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

(19) United States
(12) Patent Application Publication
(10) Pub. No.: US 2023/0414668 A1
(43) Pub. Date: Dec. 28, 2023

(54) CELL-BASED THERAPY FOR VIRAL DISEASES

(71) Applicant: OTTAWA HOSPITAL RESEARCH INSTITUTE, Ottawa (CA)

(72) Inventors: Shirley H.J. MEI, Ottawa (CA); Luciana DE SOUZA MOREIRA, Ottawa (CA); Yuan TAN, Ottawa (CA); Yan WANG, Ottawa (CA)

(21) Appl. No.: 18/249,219

(22) PCT Filed: Oct. 15, 2021
(86) PCT No.: PCT/CA2021/051448

§ 371 Date: Apr. 14, 2023

Related U.S. Application Data
(60) Provisional application No. 63/092,572, filed on Oct. 16, 2020.

(51) Int. Cl. A61K 35/28; C07K 14/515; C07K 14/555; A61P 31/16

(52) U.S. Cl. CPC A61K 35/28; A61P 31/16; C07K 14/555; C07K 14/515

(57) ABSTRACT

The present application relates to the prevention or treatment of symptoms and diseases caused by viral infections, such as acute respiratory distress syndrome (ARDS), sepsis or septic shock associated with SARS-CoV-2 and influenza virus infections. The treatment is based on the use of mesenchymal stem cells (MSCs) genetically engineered to overexpress suitable polypeptides such as angiopoeitin-1 (ANGPT1) and IFN-induced transmembrane (IFITM). The MSC-based therapy may be administered to the lung of the patient by injection into the blood system.

### CROSS REFERENCE TO RELATED APPLICATIONS

This application claims the benefit of U.S. provisional application Ser. No. 63/092,572, filed on Oct. 16, 2020, which is incorporated herein by reference in its entirety.

### SEQUENCE LISTING

This application contains a Sequence Listing in computer readable form entitled G17018-00018-Seq Listing_ST25.txt, created on Oct. 15, 2021 having a size of −15 kilobytes, which is incorporated herein by reference.

### TECHNICAL FIELD

The present disclosure relates to methods and compositions regarding cell-based therapeutics and therapies for viral diseases such as Coronavirus disease (COVID-19).

### BACKGROUND ART

Cell-based gene transfer is a technique for conducting ex vivo modification on the cell to transfer nucleic acid sequence materials. In this procedure, nucleic acid sequences containing the genes which it is desired to introduce into the patient's body (the transgenes) are prepared extracellularly, e.g., by using molecular biology and genetic engineering technologies. Mammalian cells such as the patient's own (i.e., autologous) or cells from another individual (i.e., allogenic) cells are then cultured in vitro and treated so as to take up the transgene in an expressible form. The transgenes may be foreign to the mammalian cell, additional copies of genes already present in the cell, to increase the amount of expression product of the gene or copies of normal genes which may be defective or missing in a particular patient, or whose overexpression in the patient is desirable. Then the cells containing the transgene are introduced into the patient, so that the gene may express the required gene products in the body, for therapeutic purposes. The take-up of the foreign gene by the cells in culture may be accomplished by genetic engineering techniques, e.g., by causing transfection of the cells with a virus containing the nucleic acid of the gene to be transferred by lipofection, by electroporation, or by other accepted means to obtain transfected cells, such as the use of viral vectors. This is sometimes followed by selective culturing of the cells which have successfully taken up the transgene in an expressible form, so that administration of the cells to the patient can be limited to the transfected cells expressing the transgene. In other cases, all of the cells subject to the take-up process are administered.

This procedure has in the past required administration of the cells containing the transgene directly to the body organ requiring treatment with the expression product of the transgene. Thus, transfected cells in an appropriate medium have been directly injected into the liver or into the muscle requiring the treatment, or via the systemic arterial circulation to enter the organ requiring treatment.

Previous attempts to introduce such genetically modified cells into the systemic arterial circulation of a patient have encountered a number of problems. For example, there is difficulty in ensuring a sufficiently high assimilation of the genetically modified cells by the specific organ or body part where the gene expression product is required for best therapeutic benefit. This lack of specificity leads to the administration of excessive amounts of the genetically modified cells, which is not only wasteful and expensive, but also increases risks of side effects.

On Mar. 11, 2020, the World Health Organization declared Coronavirus disease (COVID-19) as a pandemic (1). COVID-19 is caused by a novel coronavirus, Severe Acute Respiratory Syndrome Coronavirus 2 (SARS-CoV-2). Approximately 15% of hospitalized patients developed severe disease with viral pneumonia, while 6% progressed to a critical stage requiring intensive care admission (2), due to severe complications such as acute respiratory distress syndrome (ARDS), sepsis or septic shock.

The hallmark of COVID-19 infection is viral-induced acute inflammation leading to lung injury (pneumonia and ARDS), characterized by ground glass opacity radiological findings. ARDS presents in the severe cases of COVID-19, along with sepsis and septic shock, and organ injury and failure (3). In these patients, dysfunctional immune responses of the host cause a cascade of endothelial and coagulopathy responses. These states together culminate in persistent organ injury and/or even death (4). ARDS can present with other co-morbidities, such as sepsis, and may lead to organ failure and death (5). The pathogenic mechanism of ARDS is increased capillary permeability due to damage of the capillary endothelium and alveolar epithelium. This leads to a pathway where impaired fluid removal from the alveolar space causes accumulation of fluid inside the alveoli, leading to alveolar damage and the release of pro-inflammatory cytokines along with neutrophils (5). Another major complication affecting COVID-19 mortality is sepsis. Patients suffer a high mortality rate of 20-40%, and those who survive face long-term morbidity associated with physical, cognitive, and emotional dysfunction (6, 7). In 2013, sepsis accounted for more than $24 billion in hospital expenses in the U.S, with cost estimated increase to $62 billion in 2019 (8). Despite decades of clinical trials, no specific therapeutic agent has produced significant improvement in survival or long-term prognosis. Similar to ARDS, sepsis also causes widespread endothelial dysfunction, elevated procoagulant state, and increased vascular permeability, all of which cumulatively will result in persistent tissue injury, organ failure or even death (9, 10).

The pathology of COVID-19 infection has been linked to cytokine storm syndrome, whereby viral infection triggers acute release of pro-inflammatory cytokines (IL-2, IL-6, IL-7, GCSF, IP-10, MCP-1, MIP1A and TNFα (11), which can induce pulmonary edema, ARDS, acute cardiac injury, sepsis and septic shock (due to secondary infection) leading to death (11). Under the current global pandemic, COVID-19 poses a serious and immediate health threat to the US civilian and world population. Current therapies for severe COVID-19 patients in ICU are only supportive, non-specific to pathogen (ex. general antiviral therapy, antibiotics) or have potentially significant side effects (e.g., Chloroquine). Consolidated antibody therapy is currently being investigated as a possible treatment option. However, this will not address the dysfunctional host immune response that leads to adverse outcomes. Urgent need for an effective immunomodulatory therapy to address pathological immune responses in COVID-19 infection. In the absence of effective therapies, the outcome of COVID-19 infection is essentially dependent on one's own immune response.

The present description refers to a number of documents, the content of which is herein incorporated by reference in their entirety.

### SUMMARY

The present disclosure relates to the following items:
1. A mesenchymal stem cell (MSC) that is genetically modified to overexpress an angiopoeitin-1 (ANGPT1) polypeptide and an IFN-induced transmembrane (IFITM) polypeptide.
2. The MSC of item 1, wherein the MSC expresses a recombinant ANGPT1 polypeptide.
3. The MSC of item 2, wherein the recombinant ANGPT1 polypeptide comprises an amino acid sequence that is at least 70% identical to the amino acid sequence of residues 16 to 498 of SEQ ID NO:9.
4. The MSC of item 3, wherein the recombinant ANGPT1 polypeptide comprises an amino acid sequence that is at least 90% identical to the amino acid sequence of residues 16 to 498 of SEQ ID NO:9.
5. The MSC of item 4, wherein the recombinant ANGPT1 polypeptide comprises the amino acid sequence of residues 16 to 498 of SEQ ID NO:9.
6. The MSC of any one of items 1 to 5, wherein the MSC comprises a first exogenous nucleic acid comprising an ANGPT1 polypeptide-encoding region operably linked to a promoter or promoter/enhancer combination.
7. The MSC of item 6, wherein the promoter is a cytomegalovirus (CMV) promoter.
8. The MSC of item 6, wherein the promoter/enhancer combination is a CMV promoter/enhancer combination.
9. The MSC of any one of items 6 to 8, wherein the ANGPT1 polypeptide-encoding region comprises a nucleotide sequence having at least 70% identity with the nucleotide sequence of SEQ ID NO: 7 or 8.
10. The MSC of item 9, wherein the ANGPT1 polypeptide-encoding region comprises the nucleotide sequence of SEQ ID NO: 7 or 8.
11. The MSC of any one of items 1 to 10, wherein the MSC expresses a recombinant IFITM polypeptide.
12. The MSC of item 11, wherein the recombinant IFITM polypeptide is a recombinant IFITM1 or IFITM3 polypeptide.
13. The MSC of item 12, wherein the recombinant IFITM polypeptide comprises an amino acid sequence that is at least 70% identical to the amino acid sequences of SEQ ID NO: 3 or 6.
14. The MSC of item 13, wherein the recombinant IFITM polypeptide comprises an amino acid sequence that is at least 90% identical to the amino acid sequences of SEQ ID NO: 3 or 6.
15. The MSC of item 14, wherein the recombinant IFITM polypeptide comprises the amino acid sequences of SEQ ID NO: 3 or 6.
16. The MSC of any one of items 11 to 15, wherein the MSC comprises a second exogenous nucleic acid comprising an IFITM polypeptide-encoding region operably linked to a second promoter or promoter/enhancer combination.
17. The MSC of item 16, wherein the second promoter is a CMV promoter.
18. The MSC of item 16, wherein the second promoter/enhancer combination is a CMV promoter/enhancer combination.
19. The MSC of any one of items 16 to 18, wherein the IFITM polypeptide-encoding region comprises a nucleotide sequence having at least 70% identity with the sequence of SEQ ID NO:1, 2, 4 or 5.
20. The MSC of item 19, wherein the IFITM polypeptide-encoding region comprises a nucleotide sequence having at least 90% identity with the sequence of SEQ ID NO:1, 2, 4 or 5.
21. The MSC of item 20, wherein the IFITM polypeptide-encoding region comprises a nucleotide sequence of SEQ ID NO:1, 2, 4 or 5.
22. The MSC of any one of items 18 to 32, wherein the first and/or second exogenous nucleic acid(s) is/are comprised in a vector or plasmid.
23. The MSC of item 22, wherein the first and second exogenous nucleic acids are comprised in the same vector or plasmid.
24. A pharmaceutical composition comprising the MSC of any one of items 1 to 23 and a pharmaceutically acceptable excipient.
25. The MSC of any one of items 1 to 23 or the pharmaceutical composition of item 24, for use as a medicament.
26. The MSC or pharmaceutical composition for use of item 25, wherein said medicament is for the prevention or treatment of a viral disease in a subject.
27. The MSC or pharmaceutical composition for use of item 26, wherein the viral disease is COVID-19 or influenza.
28. The MSC or pharmaceutical composition for use of item 26 or 27, wherein the subject is human.
29. The MSC or pharmaceutical composition for use of any one of items 26-28, wherein the MSC are allogenic with respect to the subject.
30. The MSC or pharmaceutical composition for use of any one of items 26-28, wherein the MSC are autologous with respect to the subject.
31. The MSC or pharmaceutical composition for use of any one of items 26-30, wherein the MSC are for administration into the lungs.
32. A method for preventing or treating a viral disease in a subject, comprising administering to the subject an effective amount of the MSC of any one of items 1 to 23 or pharmaceutical composition of item 24.
33. The method of item 32, wherein the viral disease is COVID-19 or influenza.
34. The method of item 32 or 33, wherein the subject is human.
35. The method of any one of items 32-34, wherein the MSC are allogenic with respect to the subject.
36. The method of any one of items 32-34, wherein the MSC are autologous with respect to the subject.
37. The method of any one of items 32-36, wherein the MSC are administered into the lungs of the subject.
38. Use of the MSC of any one of items 1 to 23 or the pharmaceutical composition of item 24 for the manufacture of a medicament for the prevention or treatment of a viral disease in a subject.
39. Use of the MSC of any one of items 1 to 23 or the pharmaceutical composition of item 24 for the prevention or treatment of a viral disease in a subject.
40. The use of item 38 or 39, wherein the viral disease is COVID-19 or influenza.
41. The use of any one of items 38-40, wherein the subject is human.
42. The use of any one of items 38-41, wherein the MSC are allogenic with respect to the subject.
43. The use of any one of items 38-41, wherein the MSC are autologous with respect to the subject.
44. The use of any one of items 38-43, wherein the MSC are for administration into the lungs.

It is also an object of the present disclosure to treat the pulmonary effects of viral diseases such as COVID-19 by utilizing novel therapeutics, including cell therapy and cell-based gene therapy. The technology described herein is a novel ready-for-clinic, off-the-shelf cell-based therapy for treatment of ARDS caused by viral diseases such as COVID-19 through their enhanced anti-viral, anti-inflammatory and reparative capabilities addresses this treatment gap.

In one aspect of the disclosure, at least two recombinant proteins that suppress the pulmonary effects of viral diseases such as COVID-19 are co-expressed in a mesenchymal stem/stromal cell (MSC).

In one embodiment, the two proteins may be codon optimized.

In another embodiment, the two proteins may be and regulated by at least one promoter.

In another embodiment, Angiopoetin-1 (Angpt1) is one of the recombinant proteins.

In another embodiment, a second protein is an interferon induced transmembrane protein (IFITM).

In another embodiment, the second protein is IFITM1.

In another embodiment, the second protein is IFITM3.

In another embodiment, the expression of each of the at least two proteins is controlled by an independent promoter.

In another embodiment, an independent promoter is a CMV promoter.

In another embodiment, the nucleic acids encoding the at least two proteins are in the same vector.

In another aspect of the disclosure, an allogeneic mesenchymal stem cell that expresses at least two recombinant proteins that suppress the pulmonary effects of viral diseases such as COVID-19 is used to treat a subject suffering from a viral disease.

The present disclosure also provides a method for alleviating pulmonary symptoms of COVID-19 comprising administration of a transformed, allogeneic mesenchymal stem cells to the lung by injection into the pulmonary circulation of the mammalian patient.

The present disclosure also provides a COVID-19 alleviating therapeutic comprising a transformed, allogeneic mesenchymal stem cells wherein the therapeutic is delivered intra arterially or intravenously.

In one embodiment, the mesenchymal stem cell that expresses at least two recombinant proteins is delivered intra arterially or intravenously.

In another embodiment, the two proteins may be codon optimized.

In another embodiment, the two proteins may be and regulated by at least one promoter.

In another embodiment, Angpt1 is one of the proteins.

In another embodiment, a second protein is an IFITM.

In another embodiment, the second protein is IFITM1.

In another embodiment, the second protein is IFITM3.

In another embodiment, the expression of each of the at least two proteins is controlled by an independent promoter.

In another embodiment, an independent promoter is a CMV promoter.

In another embodiment, the nucleic acids encoding the at least two proteins are in the same vector.

In another aspect of the disclosure, an allogeneic mesenchymal stem cell that expresses at least two recombinant proteins that suppress the pulmonary effects of COVID-19 is a pharmaceutical composition.

In one embodiment, the pharmaceutical composition comprises a pharmaceutically acceptable carrier, excipient, or adjuvant.

In another embodiment, the two proteins may be codon optimized.

In another embodiment, the two proteins may be and regulated by at least one promoter.

In another embodiment, Angpt1 is one of the proteins.

In another embodiment, a second protein is an IFITM.

In another embodiment, the second protein is IFITM1.

In another embodiment, the second protein is IFITM3.

In another embodiment, the expression of each of the at least two proteins is controlled by an independent promoter.

In another embodiment, an independent promoter is a CMV promoter.

In another embodiment, the nucleic acids encoding the at least two proteins are in the same vector.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

The technology will now be described, by way of example only, with reference to the accompanying drawings, in which:

FIG. 1 is a graph showing that the antiviral genes IFITM1 and IFITM3 are upregulated in MSC preconditioned with dsRNA. Differences between the unmodified MSC versus preconditioned dsRNA-MSC were assessed using a two-tailed t-test with statistic software (GraphPad Prism version 8);

FIGS. 2A and 2B show the IFITM1-Angpt1 plasmid vector transgene construct and the IFITM3-Angpt1 plasmid vector transgene construct, respectively;

FIGS. 3A-E show the quantification of protein expression in engineered MSCs. FIG. 3A: absolute Angiopoietin-1 levels as determined by ELISA; FIG. 3B: absolute IFITM1 levels as determined by ELISA; FIG. 3C: absolute IFITM3 levels as determined by ELISA; FIG. 3D: relative IFITM1 expression as determined by Western Blot (unmodified=1); FIG. 3E: relative IFITM3 expression as determined by Western Blot (unmodified=1);

FIG. 3F shows IFITM3 protein expression in engineered MSC at 24 h after transfection. MSC were transfected with non-viral vector then maintained at 37° C. in a humidified incubator containing 5% CO₂ for 24 h. Cells were harvested, and assessment of protein levels in total cell lysate showed overexpression of IFITM3 protein levels compared to unmodified cells. Detection of protein expression was performed using the Jess™ protein detection device (Protein Simple, Biotechne) for automation of protein separation and immunodetection. Antibodies IFITM3 (D8E8G) XP Rabbit mouse antibody from Cell Signaling (Cat #59212) and β-actin antibody from R&D Systems (Cat #937215) were used to detect protein expression.

FIG. 4 shows the inhibition effect of conditioned media from polylC preconditioned MSC on pseudovirus SARS-COV2 entry into primary epithelial cells;

FIGS. 5A and 5B show derived conditioned media for candidate 1 (IFITM1-Angpt1) and candidate 2 (IFITM3-Angpt1) using research-grade transfection protocol with 48 hr conditioned media (FIG. 5A), and scale-up transfection/manufacturing protocols (FIG. 5B);

FIG. 5C shows that the inhibitory effect conditioned media of engineered MSC (candidate 2) on SARS-CoV-2 viral entry is abolished by increasing concentrations of IFITM3 blocking antibodies. Engineered MSCs were seeded and cultured for 48 hr to harvest conditioned media (CM). The IFTIM3 monoclonal antibody (Abnova/Thermo Fisher Scientific, clone 4C8-1B10) was serially diluted from 250 to 2000-fold (0.25 to 2 μg) before being added to the CM from engineered MSCs, which were later added to human alveolar epithelial cells for 1 day prior to being exposed to pseudovirus SARS-CoV-2 (baculoviruses pseudotyped with Spike proteins with a fluorescent reporter that expresses bright green fluorescence in the host cell nucleus). After 24 hr exposure to pseudovirus SARS-CoV-2, epithelial cells were analyzed by flow cytometry (Attune acoustic focusing cytometer, Thermo Fisher Scientific) to assess the percentage of fluorescent positive cells, which reflects the number of pseudovirus that had entered the cells.

FIGS. 6A-C show that engineered MSC protects mice from severe outcomes two days after influenza A virus (IAV) infection. In this study, eight to ten-week-old C57BL/6N mice were purchased from Charles River Laboratories (Saint Constant, QC, Canada). Animals were housed according to Canadian Council on Animal Care (CCAC) guidelines. A standard chow diet was provided within the hopper of the cage, and water was provided ad libitum. Animals were housed in a 20° C.-23° C. temperature-controlled room with a 12-hour light, 12-hour dark cycle, and 40%-60% humidity. Briefly, mice received 1000 PFU of influenza A strain H1N1: A/FM/1/47-MA virus intranasally. At day two after virus inoculation, mice were treated with vehicle or engineered MSCs (candidate 2, engineered with ANGPT1 and IFITM3). Mice were monitored daily for survival (FIG. 6A), body weight (FIG. 6B) and sickness score (FIG. 6C) until 7 days after virus infection. Animal sickness score (FIG. 6C) represents scoring based on physical appearance, including facial grimace, hunch, piloerected hair, hydration, respiration rate and respiration quality as well as behaviors, including level of consciousness, activity and response to stimulus. Score ranges from 0 to 30, with higher score indicates the deteriorating condition of the mice.

FIGS. 7A-C show that engineered MSC protects mice from severe outcomes one day after IAV infection. The study was performed as described above for FIGS. 6A-C, except that 1) the mice were treated with vehicle or engineered MSCs (candidate 2) at day one after virus inoculation, 2) survival was assessed until day 9 and 3) weight loss and sickness score were assessed until day 10. Mice were monitored daily for survival (FIG. 7A), body weight (FIG. 7B) and sickness score (FIG. 7C). Animal sickness score (FIG. 7C) represents scoring based on physical appearance, including facial grimace, hunch, piloerected hair, hydration, respiration rate and respiration quality as well as behaviors, including level of consciousness, activity and response to stimulus. Score ranges from 0 to 30, with higher score indicates the deteriorating condition of the mice.

FIGS. 8A-C shows the native nucleotide sequence (FIG. 8A, SEQ ID NO:1), codon-optimized nucleotide sequence (FIG. 8B, SEQ ID NO:2), and amino acid sequence (FIG. 8C, SEQ ID NO:3) of IFITM1;

FIG. 9 shows the native nucleotide sequence (FIG. 9A, SEQ ID NO:4), codon-optimized nucleotide sequence (FIG. 9B, SEQ ID NO:5), and amino acid sequence (FIG. 9C, SEQ ID NO:6) of IFITM3; and

FIG. 10 shows the native nucleotide sequence (FIG. 10A, SEQ ID NO:7), codon-optimized nucleotide sequence (FIG. 10B, SEQ ID NO:8), and amino acid sequence (FIG. 10C, SEQ ID NO:9) of ANGPT1.

### DETAILED DISCLOSURE

The following Detailed Description, given by way of example, but not intended to limit the disclosure to specific embodiments described, may be understood in conjunction with the accompanying figures, incorporated herein by reference.

Unless otherwise defined, all technical and scientific terms used herein have the same meaning as commonly understood by one of ordinary skill in the art to which this technology belongs (e.g., in stem cell biology, cell therapy, gene therapy, cell culture, molecular genetics, immunology, immunohistochemistry, protein chemistry, and biochemistry).

In case of conflict, the present application, including definitions will control.

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted. All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

A “carrier,” “excipient,” or “adjuvant” refers to any component of a pharmaceutical composition that is not an active ingredient.

A “subject” is a vertebrate, including any member of the class mammalia, including humans, domestic and farm animals, and zoo, sports or pet animals, such as mouse, rabbit, pig, sheep, goat, cattle and higher primates.

In the studies described herein, the preparation of mesenchymal stem cells (MSCs) that are genetically engineered to overexpress certain proteins of interest is described. More specifically, MSCs genetically engineered to overexpress ANGPT1 and an IFITM protein (IFITM1 or IFITM3), were shown to protect epithelial cells from SARS-CoV-2 pseudovirus entry, and to reduce disease severity and improve survival in an influenza A H1N1 virus-induced ARDS murine model. The results provide evidence that MSCs genetically engineered to overexpress ANGPT1 and an IFITM protein may be useful for the prevention or treatment of viral diseases, such as COVID-19 or flu, in humans.

In a first aspect, the present disclosure relates to an MSC that is genetically engineered to overexpress an ANGPT1 polypeptide and an IFITM polypeptide, such as an IFITM1 or IFITM3 polypeptide.

MSCs engineered to overexpress gene can be used to enhance modulatory effects on host immune response and target disease pathophysiology. Genetic engineering of desirable immunomodulating cells, like MSCs, represents a promising strategy with many advantages, since cells can be transfected ex vivo in culture affording a greater ability to control conditions and maximize efficacy (15). This effort involves the isolation and culture of allogeneic MSCs (from an unrelated healthy donor), which can be banked in large numbers, while the immune privileged nature of MSCs allows these cells to be transplanted to unrelated recipients without Human Leukocyte Antigens matching (16). These MSCs are then transfected (gene transfer) with a therapeutic transgene, before being injected into the venous circulation and carried to sites of inflammation where the gene product, a therapeutic protein, is expressed and/or released. Genetically engineered cell-based strategy is superior to unmodified cells, as the beneficial effects of MSCs can be enhanced by introducing different therapeutic proteins that can be directly delivered to the site of injury (i.e., the lung).

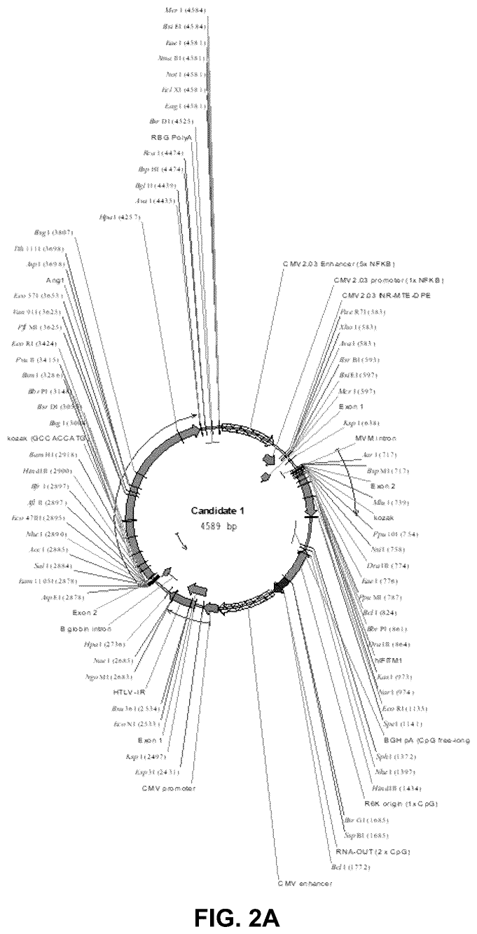

An allogeneic, genetically engineered, off-the-shelf MSC product has the potential to be administered to and treat critically ill patients in order to mitigate damage and promote repair in lungs or even multiple organs (consequence of ARDS or sepsis). The overexpression of double genes is an innovative approach that has a selective advantage to help patients to engage specific host immune responses against the virus infection. Due to multifactorial effects of the genetically engineered MSC, it is believed that the antiviral response will be accompanied by faster recovery of organ injury caused by SARS-COV-2. A plasmid with dual CMV promoters was used here to express both Angpt1 and IFITM (IFITM1 or IFITM3) genes (FIGS. 2A-2B).

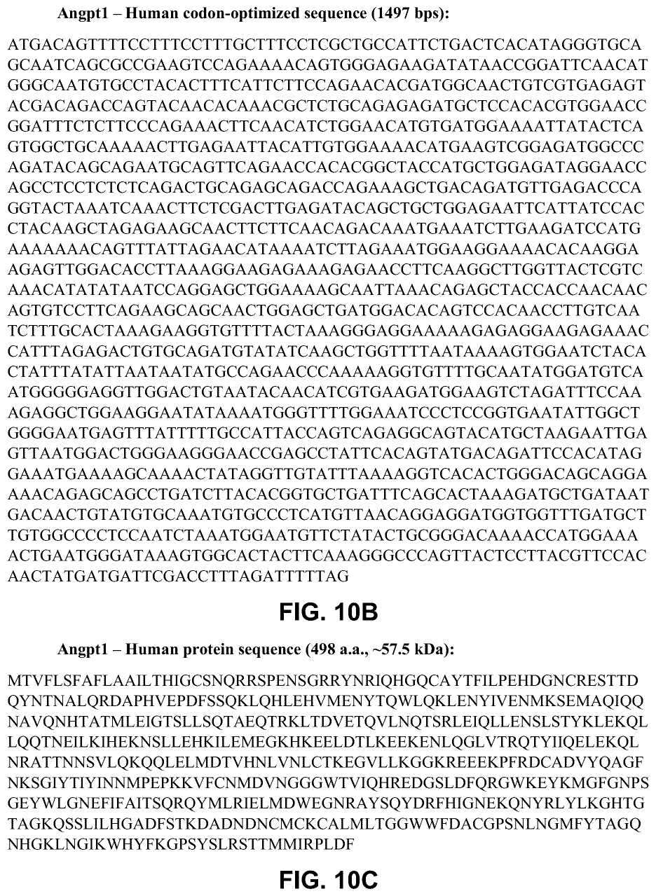

ANGPT1 is a protein of 498 amino acids in human (UniProtKB accession number Q15389, FIG. 10C) that includes an amino-terminal signal peptide of 15 amino acids. It comprises a super clustering domain encompassing residues 81-119 and a central coiled domain encompassing residues 153-261 that are involved in ANGPT1 oligomerization, and a Fibrinogen C-terminal domain encompassing residues 277-497 that is involved in the binding to its target receptor. This secreted glycoprotein binds and activates the TEK/TIE2 receptor tyrosine kinase (CD202B), expressed almost exclusively in endothelial cells, by inducing its dimerization and tyrosine phosphorylation. Mutation of position 119 (A119S) has been shown to reduce binding capability to its receptor. Mutation of position 249 (K249R) has been detected in subjects with primary congenital glaucoma (PCG) (associated with a defect in ANGPT/TEK signaling) and is believed to reduce its biological activity. Deletion of the five C-terminal amino acids (R494*) is predicted to destabilize the protein (Thomson et al., J Clin Invest. 2017 Dec. 1; 127(12): 4421-4436). Large scale protein production is challenging due to aggregation and insolubility of the protein, and recombinant ANGPT1 protein has a short half-life in vivo (21), which limit the possibility of using recombinant ANGPT1 for therapeutic applications. Such limitations may be overcome by the overexpression of ANGPT1 in MSCs according to the present disclosure.

The IFITM proteins were identified more than 25 years ago, and several IFITM orthologous genes pseudogenes were observed in genome sequences of reptile, amphibious, birds and mammals. In humans, five human IFITM genes have been identified (IFITM1, IFITM2, IFIT3, IFIM5, IFITM10) (22). IFITM1, IFITM2 and IFITM3 proteins are expressed basally in most of cell types, but this expression can be induced by virus infection and/or IFN type I and Type II (23, 24). Distinct IFITM functions have been described, such as bone mineralization, germ cell and embryonic development, tumor suppression and immune functions. The localization of IFITM1 expression has been described to be slightly different from IFITM2 and IFITM3. IFITM1 expression was demonstrated at the cell surface and in vesicular compartment different from that occupied by IFITM-2 or -3.

Human IFITM1 (UniProtKB-P13164) is a protein of 125 amino acids that comprises two cytoplasmic domains (residues 1-36 and 58-86), one intramembrane domain (residues 37-57), one transmembrane domain (residues 87-107) and one extracellular domain (residues 108-125). The membrane proximal cysteine residues at position 50, 51 and 84 may be palmitoylated, which controls clustering in membrane compartments.

Human IFITM3 (UniProtKB-Q01628) is a protein of 133 amino acids that comprises two cytoplasmic domains (residues 1-57 and 79-107), one intramembrane domain (residues 58-78), one transmembrane domain (residues 108-128) and one extracellular domain (residues 129-133). The membrane proximal cysteine residues at position 71, 72 and 105 may be palmitoylated, which controls clustering in membrane compartments. The lysine residues at positions 24 (mainly), 83, 88 and 104 may be ubiquitinated, which negatively affects IFITM3's activity.

The term “overexpress” as used herein means that the level of expression of ANGPT1 and/or the IFITM protein in the genetically-engineered MSC is higher than the level of expression of ANGPT1 and/or the IFITM protein in a corresponding MSC that does not comprise the genetic modification(s). In an embodiment, the level of expression of ANGPT1 and/or the IFITM protein in the genetically-engineered MSC is at least 20%, 50%, 100% (2-fold), 200% (3-fold), 300% (4-fold), 400% (5-fold), 900% (10-fold), 20-fold, 50-fold or 100-fold higher than the level of expression of ANGPT1 and/or the IFITM protein in a corresponding MSC that does not comprise the genetic modification.

In an embodiment, the overexpression of the ANGPT1 protein and/or the IFITM protein is achieved by increasing the expression of the endogenous ANGPT1 gene and/or the IFITM gene in the MSC. This may be achieved, for example, by introducing suitable promoter(s) and/or transcriptional regulatory (e.g., enhancer) sequences in transcriptional relationship with (i.e., operably-linked to) the endogenous ANGPT1 gene and/or of the IFITM gene. Alternatively, this may be achieved by excising/inactivating a transcriptional repressor sequence that inhibits the expression of the endogenous ANGPT1 gene and/or of the IFITM gene. Introduction of promoter(s) and/or transcriptional regulatory (e.g., enhancer) sequences and excision/inactivation of transcriptional repressor sequences to induce overexpression of endogenous genes in the MSC may be achieved using genome editing techniques. Genome editing techniques can modify gene expression in a target cell by inserting, replacing, or removing DNA in the genome using an artificially engineered nuclease. Examples of such nucleases may include zinc finger nucleases (ZPNs) (Gommans et al., J. Mol Biol, 354(3): 507-519 (2005)), transcription activator-like effector nucleases (TALENs) (Zhang et al., Nature Biotechnol, 29: 149-153 (2011)), the CRISPR/Cas system (Cheng et al., Cell Res., 23: 1163-71] (2013)), and engineered meganucleases (Riviere et al., Gene Ther., 21(5): 529-32 (2014)). The nucleases create specific double-stranded breaks (DSBs) at targeted locations in the genome, and use endogenous mechanisms in the cell to repair the induced break by homologous recombination (HR) and nonhomologous end-joining (NHEJ). Such techniques may be used to achieve overexpression of endogenous ANGPT1 and/or IFITM genes in the MSCs.

A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequence. Generally, operably-linked DNA sequences are contiguous and, where necessary to join two protein coding regions, in reading frame. However, since for example enhancers generally function when separated from the promoters by several kilobases and intronic sequences may be of variable lengths, some nucleic acids may be operably-linked but not contiguous. “Transcriptional regulatory sequences” or “transcriptional regulatory elements” are generic terms that refer to DNA sequences, such as initiation and termination signals, enhancers, and promoters, splicing signals, polyadenylation signals, etc., which induce or control transcription of protein coding sequences with which they are operably-linked.

In another embodiment, the overexpression of the ANGPT1 protein and/or the IFITM protein is achieved by inducing the expression of a recombinant (i.e., exogenous) ANGPT1 polypeptide and/or a recombinant IFITM polypeptide in the MSC. Thus, in another aspect, the present disclosure relates to a genetically-engineered MSC comprising or overexpressing a recombinant (i.e., exogenous) ANGPT1 polypeptide. In an embodiment, the genetically-engineered MSC further comprises or overexpresses a recombinant (i.e., exogenous) IFITM polypeptide, such as a recombinant IFITM1 or IFITM3 polypeptide.

The term “recombinant” means that something has been recombined, so that when made in reference to a nucleic acid, the term refers to a molecule that is comprised of nucleic acid sequences that are joined together or produced by means of molecular biological techniques. The term “recombinant” when made in reference to a protein or a polypeptide refers to a protein or polypeptide molecule that is not synthesized from the native genome of the cell, e.g., which is expressed from a recombinant nucleic acid construct created by means of molecular biological techniques (whether or not this synthetic nucleic acid construct is integrated in the genome of the cell). Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as “recombinant” therefore indicates that the nucleic acid molecule has been manipulated using genetic engineering, i.e., by human intervention. Recombinant nucleic acid constructs may for example be introduced into a host cell by transformation (e.g., transduction or transfection). Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

In an embodiment, the recombinant (i.e., exogenous) ANGPT1 polypeptide comprises or consists of a sequence that is at least 50, 60, or 70% identical to the amino acid sequence of mature native human ANGPT1 depicted in FIG. 10C (residues 16-498) and that exhibits the biological activity of native human ANGPT1, e.g., the ability to bind and activate the TEK/TIE2 receptor. In embodiments, the recombinant (i.e., exogenous) ANGPT1 polypeptide comprises or consists of a sequence that is at least 75%, 80%, 85%, 90%, 95%, 96%, 97%, 98% or 99% identical to the amino acid sequence of mature native human ANGPT1 depicted in FIG. 10C (residues 16-498) and that exhibits the biological activity of native human ANGPT1. In an embodiment, the recombinant (i.e., exogenous) ANGPT1 polypeptide comprises or consists of a sequence that is at least 70% identical to the amino acid sequence of full-length native human ANGPT1 depicted in FIG. 10C (residues 1-498) and that exhibits the biological activity of native human ANGPT1. In embodiments, the recombinant (i.e., exogenous) ANGPT1 polypeptide comprises or consists of a sequence that is at least 75%, 80%, 85%, 90%, 95%, 96%, 97%, 98% or 99% identical to the amino acid sequence of full-length native human ANGPT1 depicted in FIG. 10C (residues 1-498) and that exhibits the biological activity of native human ANGPT1.

Thus, the recombinant (i.e., exogenous) ANGPT1 polypeptide may be the native human ANGPT1 protein (full-length or mature form) or a biologically active variant thereof comprising amino acid deletions, substitutions and/or additions that do not significantly interfere with the biological activity. In an embodiment, the variant exhibits a biological activity that is at least 70%, 75%, 80%, 85%, 90%, 95% or 100% of that of native human ANGPT1. In an embodiment, the variant does not comprise a mutation in a region involved in ANGPT1 oligomerization, for example a mutation in the super clustering domain (residues 81-119) or the central coiled domain (residues 153-261). In another embodiment, the variant does not comprise a mutation in the fibrinogen C-terminal domain (residues 277-497). In further embodiments, the variant does not comprise a mutation of position 119 (e.g., A119S) and/or position 249 (e.g., K249R). In another embodiment, the variant does not comprise a deletion of the five C-terminal amino acids.

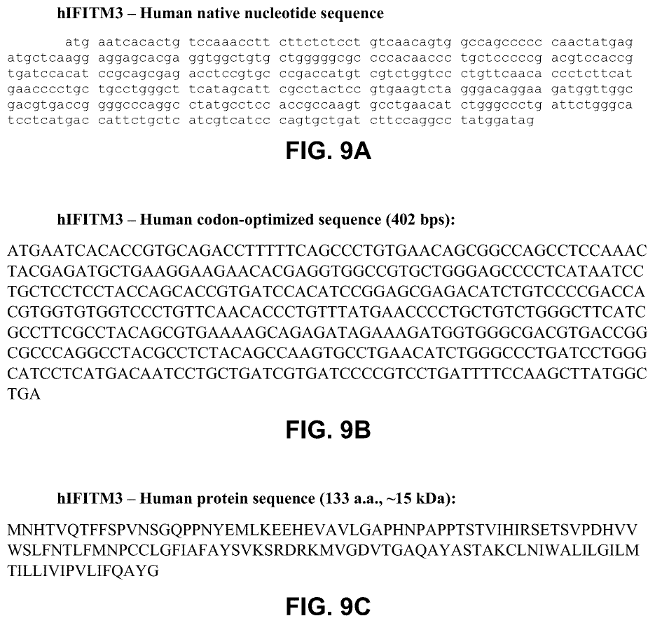

In an embodiment, the recombinant (i.e., exogenous) IFITM1 polypeptide comprises or consists of a sequence that is at least 50, 60, or 70% identical to the amino acid sequence of native human IFITM1 depicted in FIG. 8C and that exhibits the biological activity of native human IFITM1. In embodiments, the recombinant (i.e., exogenous) IFITM1 polypeptide comprises or consists of a sequence that is at least 75%, 80%, 85%, 90%, 95%, 96%, 97%, 98% or 99% identical to the amino acid sequence of native human IFITM1 depicted in FIG. 8C and that exhibits the biological activity of native human IFITM1.

Thus, the recombinant (i.e., exogenous) IFITM1 polypeptide may be the native human IFITM1 protein (full-length) or a biologically active variant thereof comprising amino acid deletions, substitutions and/or additions that do not significantly interfere with the biological activity. In an embodiment, the variant exhibits a biological activity that is at least 70%, 75%, 80%, 85%, 90%, 95% or 100% of that of native human IFITM1. In an embodiment, the variant does not comprise a mutation in one or more of the cysteine residues involved in palmitoylation of IFITM1, e.g., residues 50, 51 and/or 84.

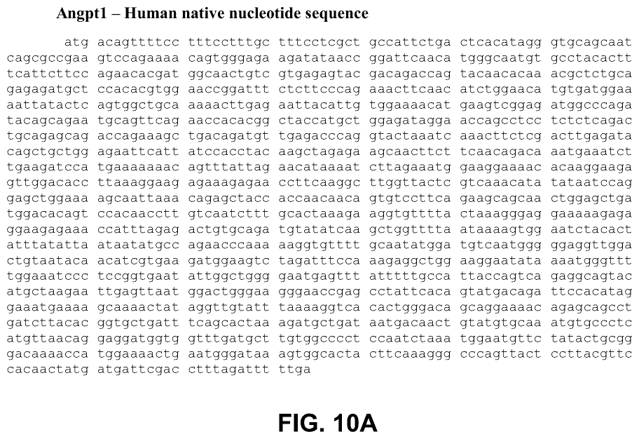

In an embodiment, the recombinant (i.e., exogenous) IFITM3 polypeptide comprises or consists of a sequence that is at least 50, 60, or 70% identical to the amino acid sequence of native human IFITM3 depicted in FIG. 9C and that exhibits the biological activity of native human IFITM3. In embodiments, the recombinant (i.e., exogenous) IFITM3 polypeptide comprises or consists of a sequence that is at least 75%, 80%, 85%, 90%, 95%, 96%, 97%, 98% or 99% identical to the amino acid sequence of native human IFITM3 depicted in FIG. 9C and that exhibits the biological activity of native human IFITM3.

Thus, the recombinant (i.e., exogenous) IFITM3 polypeptide may be the native human IFITM3 protein (full-length) or a biologically active variant thereof comprising amino acid deletions, substitutions and/or additions that do not significantly interfere with the biological activity. In an embodiment, the variant exhibits a biological activity that is at least 70%, 75%, 80%, 85%, 90%, 95% or 100% of that of native human IFITM3. In an embodiment, the variant does not comprise a mutation in one or more of the cysteine residues involved in palmitoylation of IFITM1, e.g., residues 71, 72 and/or 105. In another embodiment, the variant comprises a mutation at one or more of the residues that are ubiquitinated, e.g., residues 24, 83, 88 and/or 104.

In an embodiment, the MSC is transformed or transfected by a recombinant nucleic acid encoding the recombinant (i.e., exogenous) ANGPT1 polypeptide. Thus, in another aspect, the present disclosure relates to a genetically-engineered MSC comprising a recombinant (i.e., exogenous) nucleic acid encoding an ANGPT1 polypeptide.

As used herein, “nucleic acid” shall mean any nucleic acid molecule, including, without limitation, DNA, RNA and hybrids or modified variants thereof. An “exogenous” or “recombinant” nucleic acid relates to any nucleic acid introduced into the cell, which is not a component of the cells “original” or “natural” genome. Exogenous nucleic acids may be integrated or non-integrated, or relate to stably transfected nucleic acids.

The recombinant nucleic acid may be a DNA or mRNA molecule comprising a nucleotide sequence encoding the recombinant (i.e., exogenous) ANGPT1 polypeptide and/or the recombinant IFITM polypeptide. Methods for introducing recombinant nucleic acids into cell are well known in the art. Examples of such methods include, but are not limited to, the use of a lipid, protein, particle, or other molecule capable of facilitating cell transformation with the nucleic acid, electroporation. However, an MSC also can be contacted with a nucleic acid encoding the ANGPT1 polypeptide and/or the IFITM polypeptide in vivo, such as by way of a gene gun, for example.

In an embodiment, the recombinant nucleic acid encoding ANGPT1 comprises a nucleotide sequence that is at least 70% identical to the nucleotide sequence encoding the mature full-length human ANGPT1 depicted in FIG. 10A. In embodiments, the recombinant nucleic acid comprises a nucleotide sequence that is at least 75%, 80%, 85%, 90%, 95%, 96%, 97%, 98% or 99% identical to the nucleotide sequence encoding the full-length mature form of human ANGPT1 depicted in FIG. 10A.

In an embodiment, the nucleic acid encoding ANGPT1 comprises a codon-optimized nucleotide sequence encoding human ANGPT1, for example the codon-optimized sequence depicted in FIG. 10B. Without being bound to any particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency. Methods, tools, platforms and services to synthesize codon-optimized nucleic acids are well known in the art, such as the OptimumGene™ platform from GenScript, the Codon Optimization Tool from Integrated DNA Technologies, the GeneOptimizer™ platform from ThermoFisher Scientific, OPTIMIZER from the NCBI (Nucleic Acids Res. 2007 July; 35 (Web Server issue): W126-W131), Codon Optimization OnLine (COOL) (Bioinformatics, Volume 30, Issue 15, 1 Aug. 2014, Pages 2210-2212), and the like.

In an embodiment, the recombinant nucleic acid encoding IFITM1 comprises a nucleotide sequence that is at least 70% identical to the nucleotide sequence encoding the full-length human IFITM1 depicted in FIG. 8A. In embodiments, the recombinant nucleic acid comprises a nucleotide sequence that is at least 75%, 80%, 85%, 90%, 95%, 96%, 97%, 98% or 99% identical to the nucleotide sequence encoding the full-length of human IFITM1 depicted in FIG. 8A. In an embodiment, the nucleic acid encoding IFITM1 comprises a codon-optimized nucleotide sequence encoding human IFITM1, for example the codon-optimized sequence depicted in FIG. 8B.

In an embodiment, the recombinant nucleic acid encoding IFITM3 comprises a nucleotide sequence that is at least 70% identical to the nucleotide sequence encoding the full-length human IFITM3 depicted in FIG. 9A. In embodiments, the recombinant nucleic acid comprises a nucleotide sequence that is at least 75%, 80%, 85%, 90%, 95%, 96%, 97%, 98% or 99% identical to the nucleotide sequence encoding the full-length of human IFITM3 depicted in FIG. 9A. In an embodiment, the nucleic acid encoding IFITM3 comprises a codon-optimized nucleotide sequence encoding human IFITM3, for example the codon-optimized sequence depicted in FIG. 9B.

The recombinant nucleic acid may be a cDNA or mRNA molecule.

In an embodiment, the recombinant nucleic acid is present in a vector or plasmid, i.e. a recombinant expression vector or plasmid. Thus, in another aspect, the present disclosure relates to a genetically-engineered MSC comprising a recombinant expression vector comprising the nucleic acid encoding an ANGPT1 polypeptide as described herein. In an embodiment, the genetically-engineered MSC further comprises a recombinant expression vector comprising the nucleic acid encoding an IFITM polypeptide as described herein. In an embodiment, the nucleic acids encoding ANGPT1 and the IFITM polypeptide are present in different recombinant expression vectors. In another embodiment, the nucleic acids encoding ANGPT1 and the IFITM polypeptide are present in the same recombinant expression vector.

The recombinant expression vector or plasmid can be any suitable recombinant expression vector or plasmid that contains a recombinant nucleic acid as described above, Suitable vectors include those designed for expression of a gene of interest in MCSs, which are well known in the art. In an embodiment, the recombinant expression vector or plasmid is a miniplasmid. Miniplasmids or nanoplasmids are small (˜4 kb) circular plasmid derivatives that have been freed from all prokaryotic vector parts (i.e., they contain no bacterial DNA sequences) that used as transgene carriers for the genetic modification of mammalian cells. Miniplasmids and uses thereof are described, for example, in Minicircle and Miniplasmid DNA Vectors: The Future of Nonviral and Viral Gene Transfer, by Dr. Martin Schleef (editor), May 2013, Wiley-Blackwell, 258 pages.

In some embodiments, the recombinant expression vector is a viral vector. Genetically modified viruses have been widely applied for the delivery of genes into stem cells including MSCs. Preferred viral vectors for genetic modification of the MSCs described herein relate to retroviral vectors, in particular to gamma retroviral vectors. The gamma retrovirus (sometimes referred to as mammalian type C retroviruses) is a sister genus to the lentivirus clade, and is a member of the Orthoretrovirinae subfamily of the retrovirus family. The Murine leukemia virus (MLV or MuLV), the Feline leukemia virus (FeLV), the Xenotropic murine leukemia virus-related virus (XMRV) and the Gibbon ape leukemia virus (GALV) are members of the gamma retrovirus genus. A skilled person is aware of the techniques required for utilization of gamma retroviruses in genetic modification of MSCs. For example, the vectors described Maetzig et al. (Gamma retroviral vectors: biology, technology and application, 2001, Viruses 3(6):677-713) or similar vectors may be employed. For example, the Murine Leukemia Virus (MLV), a simple gammaretrovirus, can be converted into an efficient vehicle of genetic therapeutics in the context of creating gamma retrovirus-modified MSCs and expression of a therapeutic transgene from said MSCs after delivery to a subject. Genetically modified viruses have been widely applied for the delivery of genes into stem cells. Adenoviruses may be applied, or RNA viruses such as Lentiviruses, or other retroviruses. Adenoviruses have been used to generate a series of vectors for gene transfer cellular engineering. The initial generation of adenovirus vectors were produced by deleting the E1 gene (required for viral replication) generating a vector with a 4 kb cloning capacity. An additional deletion of E3 (responsible for host immune response) allowed an 8 kb cloning capacity. Further generations have been produced encompassing E2 and/or E4 deletions. Lentiviruses are members of Retroviridae family of viruses (M. Scherr et al., Curr Gene Ther. 2002 February; 2(1):45-55). Lentivirus vectors are generated by deletion of the entire viral sequence with the exception of the LTRs and cis acting packaging signals. The resultant vectors have a cloning capacity of about 8 kb. One distinguishing feature of these vectors from retroviral vectors is their ability to transduce dividing and non-dividing cells as well as terminally differentiated cells.

The recombinant expression vector or plasmid can comprise regulatory sequences, such as transcription and translation initiation and termination codons, which are specific to the type of host (e.g., an animal such as a human) into which the vector is to be introduced, as appropriate, and taking into consideration whether the vector is DNA- or RNA-based. The recombinant expression vector or plasmid can include one or more marker genes, which allow for selection of transformed or transfected hosts. Marker genes include biocide resistance, e.g., resistance to antibiotics, heavy metals, etc., complementation in an auxotrophic host to provide prototrophy, and the like, Suitable marker genes for the recombinant expression vectors include, for instance, neomycin G418 resistance genes, hygromycin resistance genes, tetracycline resistance genes, and ampicillin resistance genes, and the like. The recombinant expression vector or plasmid can comprise a native or normative promoter operably linked to the nucleic acid encoding ANGPT1 and/or IFITM polypeptide, and that is functional in MSCs. The selection of a promoter, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, a Rous sarcoma virus (RSV) promoter, a promoter found in the long-terminal repeat of the murine stem cell virus, the elongation factor 1 (EF1) promoter, the chicken β-actin promoter, the β-actin promoter from other species, the elongation factor-1 α (EF1 α) promoter, the phosphoglycerokinase (PGK) promoter, the human serum albumin (SA) promoter, the α-1 antitrypsin (AAT) promoter, the thyroxine binding globulin (TBG) promoter, the cytochrome P450 2E1 (CYP2E1) promoter, etc. The vectors may also utilize combination promoters such as the chicken β-actin/CMV enhancer (CAG) promoter, the human or murine CMV-derived enhancer elements combined with the elongation factor 1α (EF1α) promoters, CpG free versions of the human or murine CMV-derived enhancer elements combined with the elongation factor 1a (EF1α) promoters, the albumin promoter combined with an α-fetoprotein MERII enhancer, etc., or the diversity of tissue specific or inducible promoters know in the art such as the muscle specific promoters muscle creatine kinase (MCK), and C5-12 or the liver-specific promoter apolipoprotein A-I (ApoAI). The orientation of the various vector-encoded elements may be changed relative to each other. The promoter may comprise various motifs that enhance the binding of transcription factors such as initiator (INR), motif ten element (MTE) and downstream promoter element (DPE) motifs (see, e.g., Martinez, Transcription. 2012; 3(6): 295-299). In an embodiment, the promoter is a CMV promoter, for example a promoter comprising a sequence having at least 70, 75, 80, 85, 90, 95, 96, 97, 98, 99 or 100% sequence identity with the sequence of SEQ ID NO:10. In an embodiment, the vector further comprises a transcription enhancer. In a further embodiment the enhancer is a CMV enhancer, for example an enhancer comprising a sequence having at least 70, 75, 80, 85, 90, 95, 96, 97, 98, 99 or 100% sequence identity with the sequence of SEQ ID NO:11. In a further embodiment, the vector comprises a CMV promoter/enhancer combination comprising a sequence having at least 70, 75, 80, 85, 90, 95, 96, 97, 98, 99 or 100% sequence identity with the sequence of SEQ ID NO:12.

Likewise, the vectors could utilize a diversity of polyA signals known in the art, for example the bovine growth hormone, SV40 early or SV40 late polyA signals.

The recombinant expression vector can be designed for either transient expression, for stable expression, or for both. Also, the recombinant expression vectors can be made for constitutive expression or for inducible expression. The vectors useful in the context of the disclosure can be “naked” nucleic acid vectors (i.e., vectors having little or no proteins, sugars, and/or lipids encapsulating them), or vectors complexed with other molecules. Other molecules that can be suitably combined with the vectors include without limitation viral coats, cationic lipids, liposomes, polyamines, gold particles, and targeting moieties such as ligands, receptors, or antibodies that target cellular molecules.

Any given gene delivery method is encompassed by the disclosure and preferably relates to viral or non-viral vectors as described above, as well as biological or chemical methods of transfection. The methods can yield either stable or transient gene expression in the system used. Non-viral methods include conventional plasmid transfer and the application of targeted gene integration through the use of genome editing technologies, as described above. These represent approaches for vector transformation that have the advantage of being both efficient, and often site-specific in their integration. Physical methods to introduce vectors into cells are known to a skilled person. One example relates to electroporation, which relies on the use of brief, high voltage electric pulses which create transient pores in the membrane by overcoming its capacitance. One advantage of this method is that it can be utilized for both stable and transient gene expression in most cell types. Alternative methods relate to the use of liposomes, protein transduction domains or other suitable reagents promoting nucleic acid entry in a cell. Appropriate methods are known to a skilled person and are not intended as limiting embodiments of the present disclosure.

Mesenchymal stem cells (MSCs, also referred to as mesenchymal stromal cells) are cells found in bone marrow, blood, dental pulp cells, adipose tissue, skin, spleen, pancreas, brain, kidney, liver, heart, retina, brain, hair follicles, intestine, lung, lymph node, thymus, bone, ligament, tendon, skeletal muscle, dermis, and periosteum. MSC are capable of differentiating into different germ lines such as mesoderm, endoderm, and ectoderm. Thus, MSCs are capable of differentiating into a large number of cell types including, but not limited to, adipose, osseous, cartilaginous, elastic, muscular, and fibrous connective tissues. The specific lineage-commitment and differentiation pathway entered into by MSCs depends upon various influences, including mechanical influences and/or endogenous bioactive factors, such as growth factors, cytokines, and/or local microenvironmental conditions established by host tissues. MSCs are thus non-hematopoietic progenitor cells that divide to yield daughter cells that are either stem cells or are precursor cells that in time will irreversibly differentiate to yield a phenotypic cell. Examples of MSCs include mesenchymal precursor cells (MPCs).

MSCs are known to exhibit immune evasive properties after administration to a patient. MSCs have been shown to exhibit a beneficial immune modulatory effect in cases of transplantation of allogeneic donor material (Le Blanc et al., Lancet 2004: 363, p. 1439), thereby reducing a potentially pathogenic alloreactivity and rejection. The therapeutic delivery of MSCs can be performed via systemic injection, followed by MSC homing to and engraftment within sites of injury (Kidd et al., Stem Cells 2009: 27, p. 2614).

In some aspects, the MSCs can be progeny cells (which can also be referred to as expanded cells). Progeny cells can be produced from the in vitro culture of stem cells. Expanded cells of the disclosure may have a wide variety of phenotypes depending on the culture conditions (including the number and/or type of stimulatory factors in the culture medium), the number of passages and the like. In certain embodiments, the progeny cells are obtained after about 2, about 3, about 4, about 5, about 6, about 7, about 8, about 9, or about 10 passages from the parental population. However, the progeny cells may be obtained after any number of passages from the parental population. And the progeny cells can be obtained by culturing in a suitable culture medium.

In one embodiment, the progeny cells are multipotential expanded MSC progeny (MEMPs) as described in WO 2006/032092. Methods for preparing enriched populations of MSC from which progeny may be derived are described in WO 01/04268 and WO 2004/085630. In an in vitro context, MSCs will rarely be present as an absolutely pure preparation and will generally be present with other cells that are tissue-specific committed cells (TSCCs). WO 01/04268 refers to harvesting such cells from bone marrow at purity levels of about 0.1% to 90%. The population comprising MSC from which progeny are derived may be directly harvested from a tissue source, or alternatively it may be a population that has already been expanded ex vivo.

For example, the progeny may be obtained from a harvested, unexpanded, population of substantially purified MSC, comprising at least about 0.1, 1, 5, 10, 20, 30, 40, 50, 60, 70, 80 or 95% of total cells of the population in which they are present. This level may be achieved, for example, by selecting for cells that are positive for at least one marker typically expressed on MSCs such as CD29, CD44, CD90, CD49a-f, CD51, CD73 (SH3), CD105 (SH2), CD106, CD166, and Stro-1, and/or negative for markers such as CD45, CD34, CD14 or CD11b, CD79a or CD19 and HLA-DR.

For example, the progeny may be obtained from a harvested, unexpanded, population of substantially purified MSC, comprising at least about 0.1, 1, 5, 10, 20, 30, 40, 50, 60, 70, 80 or 95% of total cells of the population in which they are present. This level may be achieved, for example, by selecting for cells that are positive for at least one marker selected from CD29, CD44, CD90, CD49a-f, CD51, CD73 (SH3), CD105 (SH2), CD106, CD166, and Stro-1.

The MSC starting population may be derived from any one or more tissue types set out in WO 01/04268 or WO 2004/085630, namely bone marrow, dental pulp cells, adipose tissue and skin, or perhaps more broadly from adipose tissue, teeth, dental pulp, skin, liver, kidney, heart, retina, brain, hair follicles, intestine, lung, spleen, lymph node, thymus, pancreas, bone, ligament, bone marrow, tendon, and skeletal muscle.

MEMPS can be distinguished from freshly harvested MSCs in that they are positive for the marker Stro-1 and negative for the marker Alkaline phosphatase (ALP). In contrast, freshly isolated MSCs are positive for both Stro-1 and ALP. In a preferred embodiment of the present disclosure, at least 15%, 20%, 30%, 40%, 50%, 60%, 70%, 80%, 90% or 95% of the administered cells have the phenotype Stro-1⁺, ALP-.

Reference to a cell “positive” (also “+”) for a given marker means that it may be either a low (lo or dim) or a high (bright, bri) expresser of that marker depending on the degree to which the marker is present on the cell surface, where the terms relate to intensity of fluorescence or other color used in the color sorting process of the cells. The distinction of lo (or dim or dull) and bri will be understood in the context of the marker used on a particular cell population being sorted. Reference to a cell as being “negative” (or “−”) for a given marker, does not mean that the marker is not expressed at all by that cell. It means that the marker is expressed at a relatively very low level by that cell, and that it generates a very low signal when detectably labeled.

In one embodiment, the cells are taken from a patient with a viral disease (e.g., COVID-19), or a subject not suffering from a viral disease, genetically-engineered to overexpress ANGPT1 and an IFITM polypeptide, and optionally cultured in vitro using standard techniques, and administered to a patient in need of treatment as an autologous or allogeneic transplant. In an alternative embodiment, cells of one or more of the established human cell lines are used. In another useful embodiment of the disclosure, cells of a non-human animal (or if the patient is not a human, from another species) are used.

The present technology can be practiced using cells from any non-human animal species, including but not limited to non-human primate cells, ungulate, canine, feline, lagomorph, rodent, avian, and fish cells. Primate cells with which the disclosure may be performed include but are not limited to cells of chimpanzees, baboons, cynomolgus monkeys, and any other New or Old World monkeys. Ungulate cells with which the disclosure may be performed include but are not limited to cells of bovines, porcines, ovines, caprines, equines, buffalo and bison. Rodent cells with which the disclosure may be performed include but are not limited to mouse, rat, guinea pig, hamster and gerbil cells. Examples of lagomorph species with which the disclosure may be performed include domesticated rabbits, jack rabbits, hares, cottontails, snowshoe rabbits, and pikas. Chickens (Gallus gattus) are an example of an avian species with which the disclosure may be performed.

MSCs can be stored before use. Methods and protocols for preserving and storing of eukaryotic cells, and in particular mammalian cells, are well known in the art (see, for example, Pollard, J. W. and Walker, J. M. (1997) Basic Cell Culture Protocols, Second Edition, Humana Press, Totowa, N.J.; Freshney, R. I. (2000) Culture of Animal Cells, Fourth Edition, Wiley-Liss, Hoboken, N.J.). Any method maintaining the biological activity of the isolated stem cells such as mesenchymal stem/progenitor cells, or progeny thereof, may be utilized in connection with the present disclosure. In one preferred embodiment, the cells are maintained and stored by using cryo-preservation.

MSCs can be obtained using a variety of techniques. For example, a number of cell-sorting techniques by which cells are physically separated by reference to a property associated with the cell-antibody complex, or a label attached to the antibody can be used. This label may be a magnetic particle or a fluorescent molecule. The antibodies may be cross-linked such that they form aggregates of multiple cells, which are separable by their density. Alternatively, the antibodies may be attached to a stationary matrix, to which the desired cells adhere.

A “culture medium” as used herein, encompasses (a) both a culture medium that contains the typical components used for culturing a MSC, such as amino acids, glucose, and various salts, with or without the MSC, and (b) a composition isolated from the culture medium, including a composition comprising components released from the MSC during the culturing. The culture medium may contain components that are solid, liquid, gaseous or a mixture of phases and materials. Culture medium components include, but are not limited to, agar, agarose, gelatin and collagen matrices. “Culture medium” includes material that is intended for use in a cell culture, even if it has not yet been contacted with cells. For example, a nutrient rich liquid prepared for bacterial culture can be a culture medium.

In an embodiment, the MSCs are contacted with or exposed to one or more agents during or after culture/expansion (and prior to administration/use in the subject) to confer them with a desired property, e.g., to induce the expression/secretion of anti-inflammatory mediators and/or to inhibit the expression/secretion of pro-inflammatory mediators. For example, MSCs exposed to low concentration prostacyclins (e.g., treprostinil), for example about 10 μg/mL of treprostinil, have been shown to exhibit enhanced secretion of certain anti-inflammatory mediators such as IL-10, IL-13, IDO, iNOS, HLA and TGFβ, and reduced secretion of pro-inflammatory mediators such as TNFα and IL-4, by MSCs (see, e.g., WO 2018/080990). Such exposure to the one or more agents may be for any suitable time to induce the desired effect, for example at least 6, 12, 18, 24, 36 or 48 hours.

In another aspect, the present disclosure relates to the genetically-engineered MSC described herein, or a pharmaceutical composition comprising same, for use as a medicament. In an embodiment, the medicament is for the prevention or treatment of a viral disease, such as a respiratory viral disease in a subject, for example for managing complications of such viral diseases including ARDS, sepsis and/or septic shock. The present disclosure also relates to a method for preventing or treating a viral disease, such as a respiratory viral disease, for example for managing complications of such viral diseases including ARDS, sepsis and/or septic shock, in a subject, comprising administering to a subject in need thereof an effective amount of the genetically-engineered MSC described herein, or a pharmaceutical composition comprising same. The present disclosure also relates to the use of the genetically-engineered MSC described herein, or a pharmaceutical composition comprising same, for preventing or treating a viral disease, such as a respiratory viral disease, for example for managing complications of such viral diseases including ARDS, sepsis and/or septic shock, in a subject. The present disclosure also relates to the use of the genetically-engineered MSC described herein, or a pharmaceutical composition comprising same, for the manufacture of a medicament for preventing or treating a viral disease, such as a respiratory viral disease, for example for managing complications of such viral diseases including ARDS, sepsis and/or septic shock, in a subject. Examples of viruses that may cause respiratory complications such as ARDS and pneumonia include respiratory viruses (e.g., influenza viruses, coronaviruses), as well as nosocomial viral infection with Herpesviridae, namely herpes simplex virus (HSV) and cytomegalovirus (CMV). In an embodiment, the viral disease is flu or COVID-19.

In an embodiment, the methods and uses defined herein are for the prevention, treatment and/or management of infections by the Wuhan original SARS-CoV-2 strain. In another embodiment, the methods and uses defined herein are for the prevention, treatment and/or management of infections by variants of the Wuhan original SARS-CoV-2 strain, such as the B.1.1.7 (also known as VOC-202012/01), 501Y.V2 (B.1.351), P.1 (B.1.1.28.1), or B.1.617.2 (delta) variant, as well as other variants of concern (VOC) such as B.1.429, B.1.526, B.1.525, and A.23.1 (see e.g., www.cdc.gov/coronavirus/2019-ncov/cases-updates/variant-surveillance/variant-info.html).

As used herein the terms “treating”, “treat” or “treatment” include eliminating, ameliorating, alleviating, or abating a disease or condition or one or more symptoms thereof, whether or not the disease or condition is considered to be “cured” or “healed” and whether or not all symptoms are resolved. The terms also include reducing or preventing progression of a disease or condition or one or more symptoms thereof, impeding or preventing an underlying mechanism of a disease or condition or one or more symptoms thereof, and achieving any therapeutic and/or prophylactic benefit.

As used herein the terms “preventing”, “prevent” or “prevention” include reducing the occurrence and/or severity of a disease or condition or one or more symptoms thereof in a subject (e.g., a subject at risk of suffering from viral disease or from complications thereof) relative to an untreated control subject, or delaying the onset of one or more symptoms of the disease or condition in a subject (e.g., a subject at risk of suffering from viral disease or from complications thereof) relative to the untreated control subject.

In an embodiment, the genetically-engineered MSCs described herein are present in a composition, preferably a pharmaceutical composition. Thus, in another aspect, the present disclosure provides a pharmaceutical composition comprising the genetically-engineered MSCs described herein. In an embodiment, the pharmaceutical composition comprises a therapeutically effective amount of the genetically-engineered MSCs described herein.

Pharmaceutically acceptable carriers or excipients include saline, aqueous buffer solutions, solvents and/or dispersion media. The use of such carriers or excipients are well known in the art. The solution is preferably sterile and fluid to the extent that easy syringability exists. Preferably, the solution is stable under the conditions of manufacture and storage and preserved against the contaminating action of microorganisms such as bacteria and fungi through the use of preservatives, for example, parabens, chlorobutanol, phenol, ascorbic acid, thimerosal, and the like.

Some examples of materials and solutions which can serve as pharmaceutically-acceptable carriers or excipients include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; lubricants such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH-buffered solutions; polyesters, polycarbonates and/or polyanhydrides; antioxidants; antimicrobials; and other non-toxic compatible substances employed in pharmaceutical formulations.

The pharmaceutical compositions useful for the methods of the disclosure may comprise a polymeric carrier or extracellular matrix. A variety of biological or synthetic solid matrix materials (e.g., solid support matrices, biological adhesives or dressings, and biological/medical scaffolds) are suitable for use in this disclosure. The matrix material is preferably medically acceptable for use in in vivo applications. Non-limiting examples of such medically acceptable and/or biologically or physiologically acceptable or compatible materials include, but are not limited to, solid matrix materials that are absorbable and/or non-absorbable, such as small intestine submucosa (SIS), e.g., porcine-derived (and other SIS sources); crosslinked or non-crosslinked alginate, hydrocolloid, foams, collagen gel, collagen sponge, polyglycolic acid (PGA) mesh, polyglactin (PGL) mesh, fleeces, foam dressing, bioadhesives (e.g., fibrin glue and fibrin gel) and dead de-epidermized skin equivalents in one or more layers.

Suitable polymeric carriers include porous meshes or sponges formed of synthetic or natural polymers, as well as polymer solutions. One form of matrix is a polymeric mesh or sponge; the other is a polymeric hydrogel. Natural polymers that can be used include proteins such as collagen, albumin, and fibrin; and polysaccharides such as alginate and polymers of hyaluronic acid. Synthetic polymers include both biodegradable and non-biodegradable polymers. Examples of biodegradable polymers include polymers of hydroxy acids such as polylactic acid (PLA), polyglycolic acid (PGA), and polylactic acid-glycolic acid (PLGA), polyorthoesters, polyanhydrides, polyphosphazenes, and combinations thereof. Nonbiodegradable polymers include polyacrylates, polymethacrylates, ethylene vinyl acetate, and polyvinyl alcohols.

Polymers that can form ionic or covalently crosslinked hydrogels which are malleable are used to encapsulate cells. A hydrogel is a substance formed when an organic polymer (natural or synthetic) is cross-linked via covalent, ionic, or hydrogen bonds to create a three-dimensional open-lattice structure which entraps water molecules to form a gel.

Examples of materials which can be used to form a hydrogel include polysaccharides such as alginate, polyphosphazines, and polyacrylates, which are crosslinked ionically, or block copolymers such as Pluronics™ or Tetronics™, polyethylene oxide-polypropylene glycol block copolymers which are crosslinked by temperature or pH, respectively. Other materials include proteins such as fibrin, polymers such as polyvinylpyrrolidone, hyaluronic acid and collagen.

In general, these polymers are at least partially soluble in aqueous solutions, such as water, buffered salt solutions, or aqueous alcohol solutions, that have charged side groups, or a monovalent ionic salt thereof. Examples of polymers with acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups. Examples of polymers with basic side groups that can be reacted with anions are poly(vinyl amines), poly(vinyl pyridine), poly(vinyl imidazole), and some imino-substituted polyphosphazenes. The ammonium or quaternary salt of the polymers can also be formed from the backbone nitrogens or pendant imino groups. Examples of basic side groups are amino and imino groups.

In some embodiments, the pharmaceutical composition, methods of prevention/treatment or uses can comprise at least one additional therapeutic agent. For example, the composition, methods of prevention/treatment or uses may include an analgesic to aid in treating inflammation or pain, or an anti-infective agent to prevent infection of the site treated with the composition. More specifically, non-limiting examples of useful therapeutic agents include the following therapeutic categories: analgesics, such as nonsteroidal antiinflammatory drugs, opiate agonists and salicylates; anti-infective agents, such as antihelmintics, antianaerobics, antibiotics, aminoglycoside antibiotics, antifungal antibiotics, cephalosporin antibiotics, macrolide antibiotics, miscellaneous β-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, anti-inflammatory agents, corticosteroid anti-inflammatory agents, antipruritics/local anesthetics, topical anti-infectives, antifungal topical anti-infectives, antiviral topical anti-infectives; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, gastrointestinal anti-inflammatory agents, gastrointestinal anti-inflammatory agents, antacid anti-ulcer agents, gastric acid-pump inhibitor anti-ulcer agents, gastric mucosal anti-ulcer agents, H2-blocker anti-ulcer agents, cholelitholytic agent's, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hormones and hormone modifiers, such as abortifacients, adrenal agents, corticosteroid adrenal agents, androgens, anti-androgens, immunobiologic agents, such as immunoglobulins, immunosuppressives, toxoids, and vaccines; local anesthetics, such as amide local anesthetics and ester local anesthetics; minerals; and vitamins, such as vitamin A, vitamin B, vitamin C, vitamin D, vitamin E, and vitamin K.

In an embodiment, the genetically-engineered MSCs or pharmaceutical composition comprising same described herein may be used in combination with agents used to treat SARS-CoV-2 infection and/or related diseases such as COVID-19. Such agents include antiinflammatory drugs, anti-SARS-CoV-2 neutralizing antibodies, SARS-CoV-2 vaccines, and antiviral agents.

The combination of prophylactic/therapeutic agents and/or compositions of the present disclosure may be administered or co-administered (e.g., consecutively, simultaneously, at different times) in any conventional dosage form. Co-administration in the context of the present disclosure refers to the administration of more than one therapeutic in the course of a coordinated treatment to achieve an improved clinical outcome. Such co-administration may also be coextensive, that is, occurring during overlapping periods of time. For example, a first agent may be administered to a patient before, concomitantly, before and after, or after a second active agent is administered. The agents may in an embodiment be combined/formulated in a single composition and thus administered at the same time.

Compositions useful for the methods of the present disclosure may include cell culture components, e.g., culture media including amino acids, metals, coenzyme factors, as well as small populations of other cells, e.g., some of which may arise by subsequent differentiation of the stem cells.

Compositions useful for the methods of the present disclosure may be prepared, for example, by sedimenting out the subject cells from the culture medium and re-suspending them in the desired solution or material. The cells may be sedimented and/or changed out of the culture medium, for example, by centrifugation, filtration, ultrafiltration, etc.

The skilled artisan can readily determine the amount of MSCs and optional carrier(s) in compositions and to be administered in methods of the disclosure. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. The time for sequential administrations can be ascertained without undue experimentation.

In an embodiment, the following amounts and ranges of amounts of MSCs are administered to the subject: (i) from about 1×10² to about 1×10⁸ cells/kg body weight; (ii) from about 1×10³ to about 1×10⁷ cells/kg body weight; (iii) from about 1×10⁴ to about 1×10⁶ cells/kg body weight; (iv) from about 1×10⁴ to about 1×10⁵ cells/kg body weight; (v) from about 1×10⁵ to about 1×10⁶ cells/kg body weight; (vi) from about 5×10⁴ to about 0.5×10⁵ cells/kg body weight; (vii) about 1×10³ cells/kg body weight; (viii) about 1×10⁴ cells/kg body weight; (ix) about 5×10⁴ cells/kg body weight; (x) about 1×10⁵ cells/kg body weight; (xi) about 5×10⁵ cells/kg body weight; (xii) about 1×10⁶ cells/kg body weight; and (xiii) about 1×10⁷ cells/kg body weight. Human body weights envisioned include, without limitation, about 5 kg, 10 kg, 15 kg, 30 kg, 50 kg, about 60 kg; about 70 kg; about 80 kg, about 90 kg; about 100 kg, about 120 kg and about 150 kg. The MSCs may be administered either in a bolus form, i.e., injection of all the cells during a short period of time, or it may be accomplished by a continuous administration (e.g., infusion) of small numbers of cells over a long period of time, or alternatively by administration of limited size boluses on several occasions over a period of time.

Compositions useful for the methods of the present disclosure can be administered via, inter alia, localized injection (e.g., in the lungs), including catheter administration, systemic injection, intravenous injection, or parenteral administration. When administering a therapeutic composition described herein (e.g., a pharmaceutical composition), it will generally be formulated in a unit dosage injectable form (solution, suspension, emulsion).

### EXAMPLES

The present invention is illustrated in further details by the following non-limiting examples.

### Example 1: MSCs Under Simulated Viral Infection

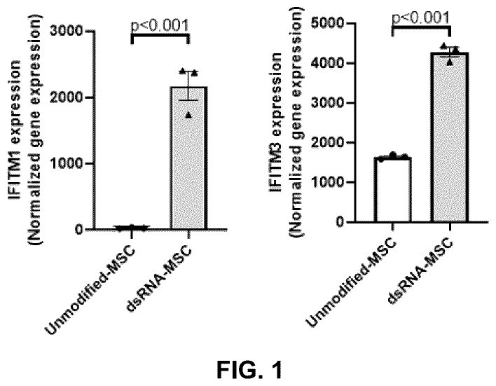

MSC were preconditioned with dsRNA that mimics a viral infection. Total transcriptome of 3 different donor-derived MSCs was analyzed with RNA sequencing performed by Qiagen®. The differential gene expression analysis showed significant upregulation of several genes related to antiviral defense, notably the IFITM1 and IFITM3 genes (FIG. 1).

### Example 2: Development of Transgene Plasmid Vectors

FIGS. 2A-B show the vector map of IFITM1-Angpt1 (FIG. 2A), and IFITM3-Angpt1 (FIG. 2B). The plasmid vectors were engineered to co-express both human codon-optimized Angpt1 and IFITM proteins under two independent CMV promoters. With plasmid vector transfection, MSCs efficiently overexpress two independent proteins from the same vector.

### Example 3: Protein Quantification

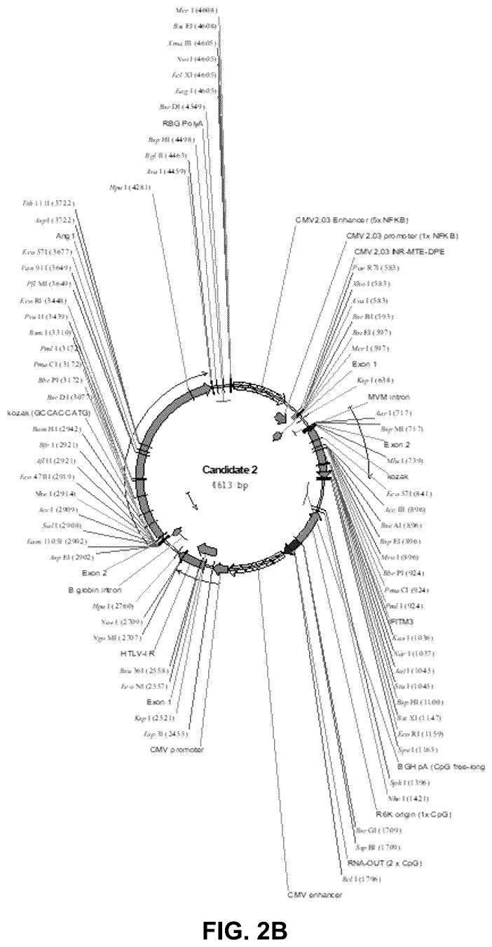

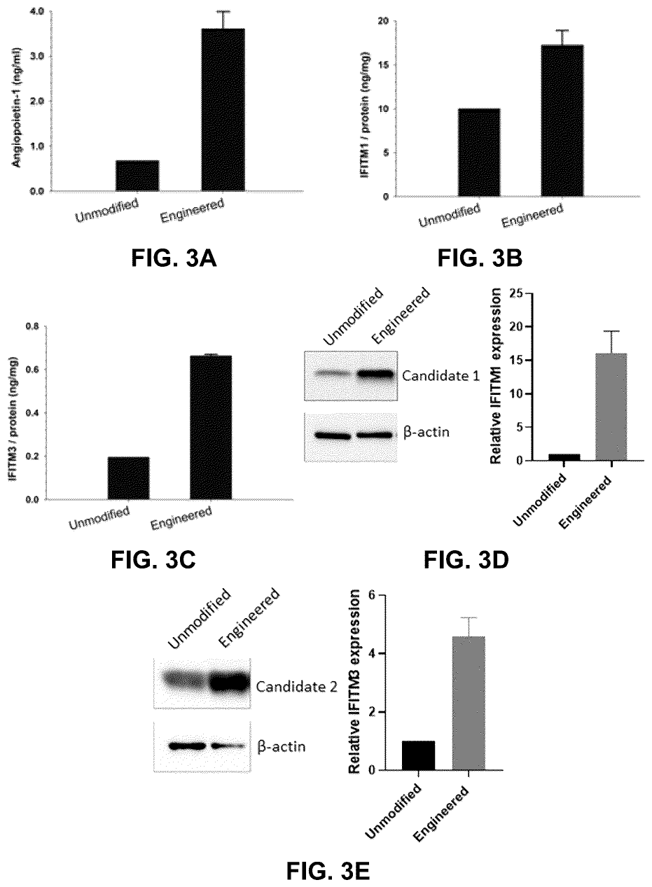

Overexpression of the proteins was verified with Western Blot and/or ELISA. In FIGS. 3A-F, MSC candidate 1 (IFITM1-Angpt1) and MSC candidate 2 (IFITM3-Angpt1) MSCs were engineered and protein expression was evaluated by ELISA (FIGS. 3A, 3B and 3C), Western Blot (FIGS. 3D, 3E) or using the Jess™ protein detection device (Protein Simple, Biotechne) (FIG. 3F). n=2 experiments.

### Example 4: Protein-Induced Protection

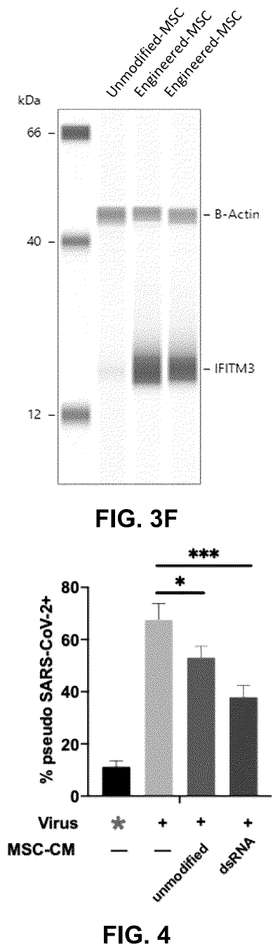

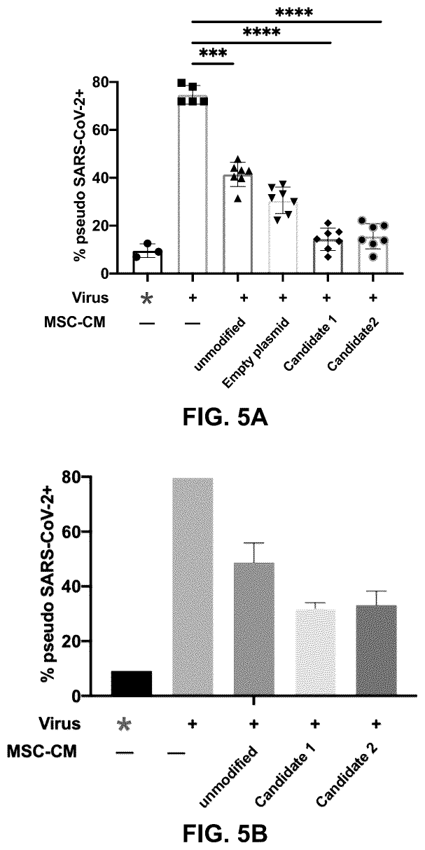

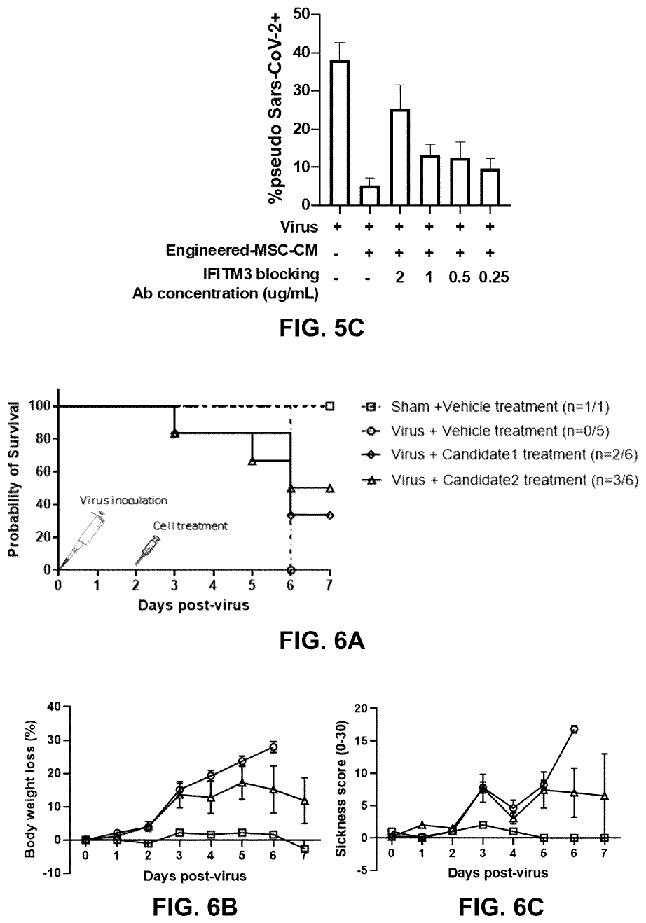

The therapeutic effect of engineered MSC was assessed using relevant potency assays to the pathophysiology of ARDS and sepsis observed in COVID-19 patients. MSC conditioned media were prepared by conditioning unmodified or modified (dsRNA stimulated or transfected cells) MSC for 24 or 48 hours, followed by a high-speed centrifugation to remove debris and dead cells. On day 0, human primary alveolar epithelial cells (ScienCell) were seeded and allowed to grow overnight for attachment. On day 1, media were replaced with MSC conditioned media mixed 1:1 with epithelial complete growth media. For data shown in FIG. 4, 5×10⁸ pseudovirus-SARS-CoV-2 (Montana Molecular) were added into cell solution on day 1, with subsequent flow cytometric analysis of green fluorescent conducted on day 2. In the prevention model (shown in FIGS. 5A-5C), pseudovirus-SARS-CoV-2 were added into cell solution on day 2 with analysis done on day 3.

Data showed that MSC candidate 1 (IFITM1-Angpt1) and MSC candidate 2 (IFITM3-Angpt1) conditioned media derived using A) research-grade transfection protocol with 48 hr conditioned media, and B) scale-up transfection/manufacturing protocol with 24 hr conditioned media protect epithelial cells from infection by pseudovirus-SARS-CoV2 induced by a coronavirus in an in vitro system. n=3 experiment done with 1-3 different donor derived MSC (bar=mean with SD). One-way ANOVA, Tukey's multiple comparison. The results depicted in FIG. 5C show that an antibody directed against IFITM3 reduces the protection from infection conferred by the conditioned media from MSC candidate 2.

### Example 5: Effect of MSC Candidate on Disease Severity and Survival in Mouse Models of ARDS (Study 1, Administration of MSC at Day 2 Post-Infection)

In vivo efficacy was assessed in a standardized murine model of ARDS and critical illness: mouse-adapted influenza A H1N1 virus (A/FM/1/47-MA virus or FM-MA virus) induced ARDS model. Eight to ten-week-old C57BL/6N mice were purchased from Charles River Laboratories (Saint Constant, QC, Canada). Animals were housed according to Canadian Council on Animal Care (CCAC) guidelines. A standard chow diet was provided within the hopper of the cage, and water was provided ad libitum. Animals were housed in a 20° C.-23° C. temperature-controlled room with a 12-hour light, 12-hour dark cycle, and 40%-60% humidity. Briefly, mice received 1000 PFU of influenza A strain H1N1: A/FM/1/47-MA virus intranasally. At day two after virus inoculation, mice were treated with vehicle or engineered MSCs (candidate 2, engineered with ANGPT1 and IFITM3). Mice were monitored daily for survival and wellness, body weight and sickness score until 7 days after virus infection.

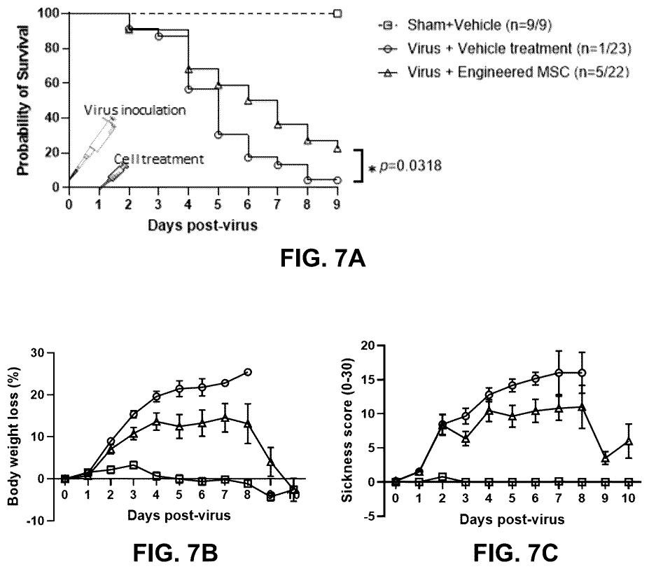

The data presented in FIG. 6A shows that the genetically engineered MSCs (candidate 2) improve the survival rate relative to vehicle-treated group (0% of vehicle group vs 50% of candidate 2 treated group at day 7) in influenza A H1N1 virus induced ARDS model. Survival rates of 33% (2/6) and 50% (3/6) were obtained using MSC candidates 1 and 2, respectively. Administration of MSC candidate 2 also partially prevented weight loss (FIG. 6B) and reduced disease severity (FIG. 6C) relative to vehicle treatment.

### Example 6: Effect of MSC Candidate on Disease Severity and Survival in Mouse Models of ARDS (Study 2, Administration of MSC at Day 1 Post-Infection)

A second study was performed using a protocol similar to that described in Example 5 above, except that 1) the mice were treated with vehicle or engineered MSCs (candidate 2) at day one after virus inoculation, 2) survival was assessed until day 9 and 3) weight loss and sickness score were assessed until day 10.

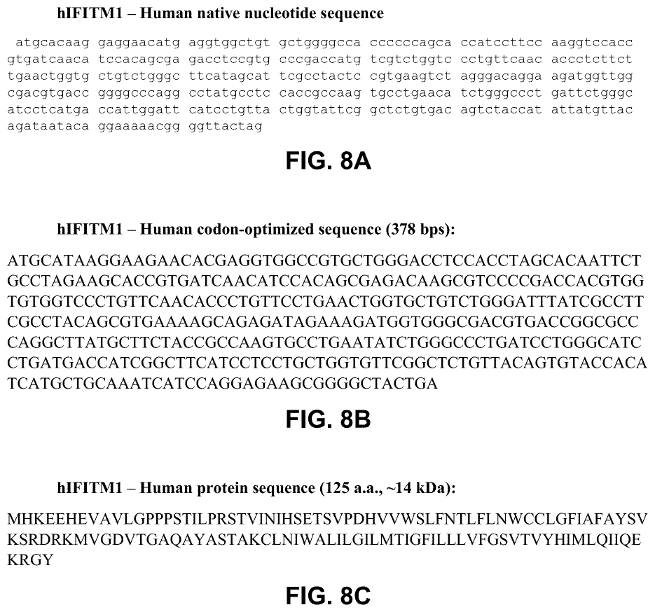

The results depicted in FIG. 7A confirm that the genetically engineered MSCs candidate 2 improve the survival rate relative to vehicle-treated group (4% of vehicle group vs. 23% of candidate 2 treated group) in influenza A H1N1 virus induced ARDS model. Administration of MSC candidate 2 also partially prevented weight loss (FIG. 7B) and reduced disease severity (FIG. 7C) relative to vehicle treatment.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. WHO. (World Health Organization, https://www.who.int/dg/speeches/detail/who-director-general-s-opening-remarks-at-the-media-briefing-on-covid-19-11-march-2020, 2020)
2. WHO. (World Health Organization, https://www.who.int/publications/item/report-of-the-who-china-joint-mission-on-coronavirus-disease-2019-(covid-19), 2020).
3. X. Yang et al., Clinical course and outcomes of critically ill patients with SARS-CoV-2 pneumonia in Wuhan, China: a single-centered, retrospective, observational study. Lancet Respir Med, (2020).
4. M. A. Matthay, R. L. Zemans, The acute respiratory distress syndrome: pathogenesis and treatment. Annu Rev Pathol 6, 147-163 (2011).
5. C. Pierrakos, M. Karanikolas, S. Scolletta, V. Karamouzos, D. Velissaris, Acute respiratory distress syndrome: pathophysiology and therapeutic options. J Clin Med Res 4, 7-16 (2012).
6. M. M. Levy et al., The Surviving Sepsis Campaign: results of an international guideline-based performance improvement program targeting severe sepsis. Intensive Care Med 36, 222-231 (2010).
7. C. E. Battle, G. Davies, P. A. Evans, Long term health-related quality of life in survivors of sepsis in South West Wales: an epidemiological study. PLoS One 9, el 16304 (2014).
8. T. G. Buchman et al., Sepsis Among Medicare Beneficiaries: 3. The Methods, Models, and Forecasts of Sepsis, 2012-2018. Crit Care Med 48, 302-318 (2020).
9. R. S. Hotchkiss et al., Sepsis and septic shock. Nat Rev Dis Primers 2, 16045 (2016).
10. C. Ince et al., The Endothelium in Sepsis. Shock 45, 259-270 (2016).
11. S. M. Metcalfe, Mesenchymal stem cells and management of COVID-19 pneumonia. Med Drug Discov 5, 100019 (2020).
12. S. Atluri, L. Manchikanti, J. A. Hirsch, Expanded Umbilical Cord Mesenchymal Stem Cells (UC-MSCs) as a Therapeutic Strategy in Managing Critically III COVID-19 Patients: The Case for Compassionate Use. Pain Physician 23, E71-E83 (2020).
13. K. Le Blanc, D. Mougiakakos, Multipotent mesenchymal stromal cells and the innate immune system. Nat Rev Immunol 12, 383-396 (2012).
14. X. Wu et al., Intrinsic Immunity Shapes Viral Resistance of Stem Cells. Cell 172, 423-438. e425 (2018).
15. S. H. Mei, C. C. Dos Santos, D. J. Stewart, Advances in Stem Cell and Cell-Based Gene Therapy Approaches for Experimental Acute Lung Injury: A Review of Preclinical Studies. Hum Gene Ther 27, 802-812 (2016).
16. J. Kurtzberg et al., Allogeneic human mesenchymal stem cell therapy (remestemcel-L, Prochymal) as a rescue agent for severe refractory acute graft-versus-host disease in pediatric patients. Biol Blood Marrow Transplant 20, 229-235 (2014).
17. J. Williams, N. T. Corporation, U.S. Pat. No. 9,550,998.
18. J. Williams, N. T. Corporation, WO 2014/077863 A1.
19. S. H. Mei et al., Prevention of LPS-induced acute lung injury in mice by mesenchymal stem cells overexpressing angiopoietin 1. PLoS Med 4, e269 (2007).
20. M. van der Heijden, G. P. van Nieuw Amerongen, S. Chedamni, V. W. M. van Hinsbergh, A. B. Johan Groeneveld, The angiopoietin-Tie2 system as a therapeutic target in sepsis and acute lung injury. Expert opinion on therapeutic targets 13, 39-53 (2009).
21. A. Alfieri et al., Angiopoietin-1 variant reduces LPS-induced microvascular dysfunction in a murine model of sepsis. Critical care (London, England) 16, R182-R182 (2012).
22. Z. Zhang, J. Liu, M. Li, H. Yang, C. Zhang, Evolutionary dynamics of the interferon-induced transmembrane gene family in vertebrates. PLoS One 7, e49265 (2012).
23. M. S. Diamond, M. Farzan, The broad-spectrum antiviral functions of IFIT and IFITM proteins. Nat Rev Immunol 13, 46-57 (2013).
24. X. Zhao, J. Li, C. A. Winkler, P. An, J. T. Guo, IFITM Genes, Variants, and Their Roles in the Control and Pathogenesis of Viral Infections. Front Microbiol 9, 3228 (2018).
25. D. C. Yánez, S. Ross, T. Crompton, The IFITM protein family in adaptive immunity. Immunology 159, 365-372 (2020).
26. I. C. Huang et al., Distinct patterns of IFITM-mediated restriction of filoviruses, SARS coronavirus, and influenza A virus. PLoS Pathog 7, e1001258 (2011).
27. G. Savidis et al., The IFITMs Inhibit Zika Virus Replication. Cell Rep 15, 2323-2330 (2016).
28. C. Kilkenny et al., Animal research: reporting in vivo experiments: the ARRIVE guidelines. Br J Pharmacol 160, 1577-1579 (2010).

1. A mesenchymal stem cell (MSC) that is genetically modified to overexpress an angiopoeitin-1 (ANGPT1) polypeptide and an IFN-induced transmembrane (IFITM) polypeptide.

2. The MSC of claim 1, wherein the MSC expresses a recombinant ANGPT1 polypeptide.

3. The MSC of claim 2, wherein the recombinant ANGPT1 polypeptide comprises an amino acid sequence that is at least 70% identical to the amino acid sequence of residues 16 to 498 of SEQ ID NO:9.

4. (canceled)

5. The MSC of claim 3, wherein the recombinant ANGPT1 polypeptide comprises the amino acid sequence of residues 16 to 498 of SEQ ID NO:9.

6. The MSC of claim 2, wherein the MSC comprises a first exogenous nucleic acid comprising an ANGPT1 polypeptide-encoding region operably linked to a promoter or promoter/enhancer combination.

7. The MSC of claim 6, wherein the promoter is a cytomegalovirus (CMV) promoter: or wherein the promoter/enhancer combination is a CMV promoter/enhancer combination.

8. (canceled)

9. The MSC of claim 6, wherein the ANGPT1 polypeptide-encoding region comprises a nucleotide sequence having at least 70% identity with the nucleotide sequence of SEQ ID NO: 7 or 8.

10. The MSC of claim 9, wherein the ANGPT1 polypeptide-encoding region comprises the nucleotide sequence of SEQ ID NO: 7 or 8.

11. The MSC of claim 1, wherein the MSC expresses a recombinant IFITM polypeptide.

12. The MSC of claim 11, wherein the recombinant IFITM polypeptide is a recombinant IFITM1 or IFITM3 polypeptide.

13. The MSC of claim 12, wherein the recombinant IFITM polypeptide comprises an amino acid sequence that is at least 70% identical to the amino acid sequences of SEQ ID NO: 3 or 6.

14. (canceled)

15. The MSC of claim 13, wherein the recombinant IFITM polypeptide comprises the amino acid sequences of SEQ ID NO: 3 or 6.

16. The MSC of claim 11, wherein the MSC comprises a second exogenous nucleic acid comprising an IFITM polypeptide-encoding region operably linked to a second promoter or promoter/enhancer combination.

17. The MSC of claim 16, wherein the second promoter is a CMV promoter: or wherein the second promoter/enhancer combination is a CMV promoter/enhancer combination.

18. (canceled)

19. The MSC of claim 16, wherein the IFITM polypeptide-encoding region comprises a nucleotide sequence having at least 70% identity with the sequence of SEQ ID NO:1, 2, 4 or 5.

20. (canceled)

21. The MSC of claim 19, wherein the IFITM polypeptide-encoding region comprises a nucleotide sequence of SEQ ID NO:1, 2, 4 or 5.

22. The MSC of claim 16, wherein the first and/or second exogenous nucleic acid(s) is/are comprised in a vector or plasmid.

23-31. (canceled)

32. A method for preventing or treating a viral disease in a subject, comprising administering to the subject an effective amount of the MSC of claim 1.

33. The method of claim 32, wherein the viral disease is COVID-19 or influenza.

34. The method of claim 32, wherein the subject is human.

35. The method of claim 32, wherein the MSC are allogenic or autologous with respect to the subject.

36. (canceled)

37. The method of claim 32, wherein the MSC are administered into the lungs of the subject.

38-44. (canceled)
